(19) United States
(12) Patent Application Publication
(10) Pub. No.: US 2015/0045803 A1
(43) Pub. Date: Feb. 12, 2015

(54) MACHINED SURGICAL GUIDE

(71) Applicants: Charles L. Deville, Asnieres sur Seine (FR); Silvie M. Bothorel, Paris (FR); Philippe Congy, Meaux (FR)

(72) Inventors: Charles L. Deville, Asnieres sur Seine (FR); Silvie M. Bothorel, Paris (FR); Philippe Congy, Meaux (FR)

(21) Appl. No.: 14/367,945

(22) PCT Filed: Jan. 6, 2012
(86) PCT No.: PCT/IB2012/000169

§ 371 Date: Oct. 8, 2014

(51) Int. Cl. A61B 17/17

(52) U.S. Cl. CPC A61B 17/17
USPC 606/96

(57) ABSTRACT

A method for forming a surgery guide for osteotomy shapes a block of material for conformance to at least a portion of the dental arch of a patient. At least one guide feature is shaped to protrude from a surface of the shaped block material. A drill guide sleeve is seated on the at least one guide feature. The shaft of a drill is guided into the sleeve and a hole drilled through the shaped block material.

### FIELD OF THE INVENTION

The invention relates generally to the field of surgical implant positioning, and more particularly to a surgical guide for drilling to position an implant within the jawbone.

### BACKGROUND OF THE INVENTION

Dental implants can be used to replace missing or badly damaged teeth. To mount a dental implant securely in bony tissue, a hole is drilled into the mandibular or jaw-bone of the patient. The implant portion that holds the artificial tooth is usually made of titanium or a titanium alloy and is able to integrate with the bone of the patient. Once the implant is seated and secure, the artificial tooth can be installed.

Osteotomy, that is the drilling of a hole in the jaw or mandibular bone at the proper angle and dimension, requires accuracy, so that the implant fits correctly without damage to surrounding tissue or structures and so that the completed work is aesthetically acceptable. For edentulous or at least partially edentulous patients, implant planning is carefully executed. Based on information from x-ray or computerized tomography (CT) imaging of the patient's dental arch, dedicated software tools allow the dentist to define the location, diameter, length or drill depth, shape and angulation of the implant to be affixed on the patient's jawbone. One consideration in this planning is reducing the risk of damage to nearby nerves or blood vessels.

One appliance that is often used to assist in implant preparation is a surgical guide. Custom-fabricated for each patient, shaped to conform to at least a portion of the patient's dental arch, the surgical guide is fitted to the patient's mouth and includes one or more guide holes to guide the dental drill down into the jawbone according to the implant planning.

The surgical guide can be fabricated as a plastic appliance using a stereolithographic process or by a milling process, based on the digital data obtained during implant planning. Some dental sites are equipped with a 4-axis milling apparatus for dental prostheses, enabling the surgical guide to be prepared on-site. Though 5-axis milling equipment is available, the 4-axis milling apparatus is less expensive and is believed suitable for use with other dental prostheses. Consequently, some dental professionals prefer a 4-axis milling apparatus to mill a surgical guide, rather than more costly equipment.

A 4-axis machine used in a conventional manner is limited in function and is unable to provide guidance holes at some angles. Referring to FIG. 1A, there are shown coordinate designations for 4-axis machining. The 4-axis milling apparatus can translate a workpiece 18 with respect to a tool 16 along each of the axes X, Y, and Z, and can rotate the workpiece about the X-axis. This allows the 4-axis milling apparatus to drill holes tilted in the YZ plane, but it cannot drill holes that are tilted in other planes. As shown in FIG. 1B, a surgical guide 10 can require drilling for guide holes 12a, 12b at angles A1 and A2 other than those in the YZ plane. Thus, there is a need for methods and apparatus that allow 4-axis milling equipment to be used for machining surgical guides, wherein drilling of the guide holes is performed separate from the milling operation.

### SUMMARY OF THE INVENTION

An object of the present invention is to advance the art of implant installation.

A related object of the present invention is to allow fabrication of a surgical guide for osteotomy using a 4-axis milling apparatus.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to one aspect of the invention, there is provided a method for forming a surgery guide for osteotomy, the method comprising: shaping a block of material for conformance to at least a portion of the dental arch of a patient; shaping at least one guide feature that protrudes from a surface of the shaped block material; seating a drill guide sleeve on the at least one guide feature; and guiding the shaft of a drill into the sleeve and drilling a hole through the shaped block material.

From an alternate aspect, the invention provides a method for forming a surgery guide for osteotomy, the method comprising: shaping a block of material for conformance to at least a portion of the dental arch of the patient, using a 4-axis milling apparatus; shaping at least one guide feature that protrudes from a surface of the shaped block material, wherein the at least one guide feature is a cylindrical structure that is tilted at an oblique angle relative to a plane that is orthogonal to an axis of rotation of the milling apparatus; seating a drill guide sleeve on the at least one guide feature, wherein the drill guide sleeve includes a seat portion that fits over the at least one guide feature and a shaft guide portion that guides a drill bit in a direction that leads into the at least one guide feature; drilling a hole through the shaped block material by a drill shaft guided into the shaft guide portion; and re-shaping the at least one guide feature.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other.

FIG. 1A shows coordinate designations used in the context of the present disclosure.

FIG. 1B shows a surgical guide with desired guide holes.

FIG. 2A is a perspective view showing an intermediate structure machined with guide features.

FIG. 2B is a perspective view of the intermediate structure of FIG. 2A with seated drill guide sleeves.

FIG. 2C is a perspective view that shows how drilled holes are formed in the surgical guide of FIG. 2B.

FIG. 2D shows the fabricated surgical guide with guide features.

FIG. 2E shows the fabricated surgical guide with guide features optionally removed.

FIG. 3 is a cross-sectional view showing how the surgical guide can be used to drill into bone tissue at proper depth and angle.

FIG. 4 is a perspective view that shows an alternate embodiment having two guide features that are very close together or are touching.

FIG. 5A is a perspective view showing a workpiece coupled to a spindle prior to machining.

FIG. 5B is a perspective view that shows an alternate embodiment using a swivel-axis workpiece for mounting the workpiece in a 4-axis machine.

FIG. 6A is a perspective view showing an alternate embodiment for mounting the workpiece in a 4-axis machine.

FIG. 6B is a plan view showing multiple partial surgical guides that can be individually positioned over local areas of the dental arch.

FIG. 6C is a perspective view that shows a surgical guide used to house multiple partial guides.

FIG. 7A is a perspective view of a milling apparatus that can be used for surgical guide fabrication in a dental office or other facility.

FIG. 7B is an exploded view, in perspective, of internal components of the milling apparatus of FIG. 7A.

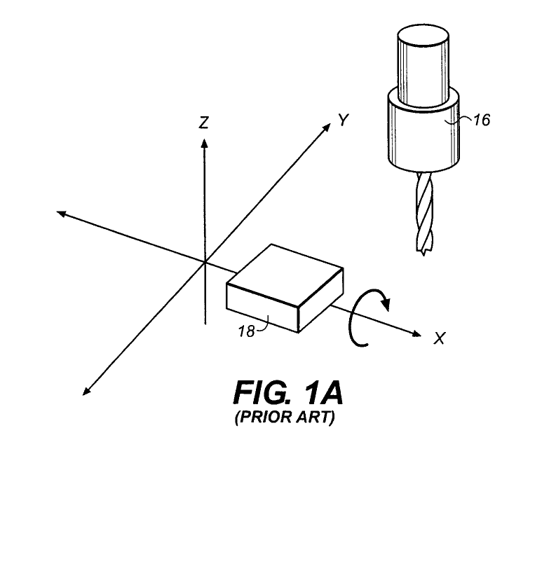

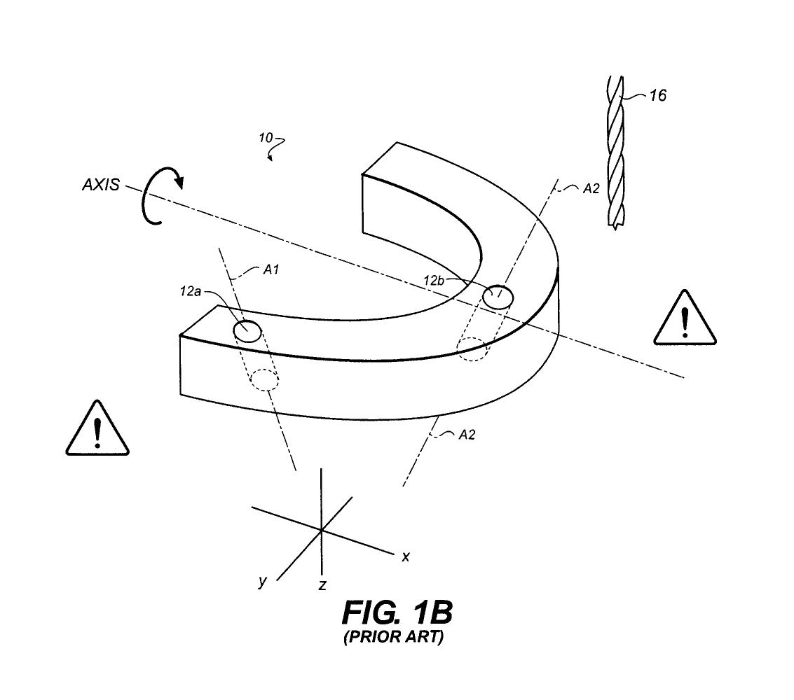

### DETAILED DESCRIPTION OF THE INVENTION

The following is a detailed description of preferred embodiments of the invention, reference being made to the drawings in which the same reference numerals identify the same elements of structure in each of the several figures. Similar descriptions concerning components and arrangement or interaction of components already described are omitted. Where they are used, the terms “first”, “second”, and so on, do not necessarily denote any ordinal or priority relation, but are simply used to more clearly distinguish one element from another.

In the context of the present disclosure, the term “oblique” describes an angular relationship wherein two lines or surfaces, or a line and a surface, are not parallel and not orthogonal, and wherein the angle between them is offset by at least more than about 5 degrees from any integer multiple of 90 degrees.

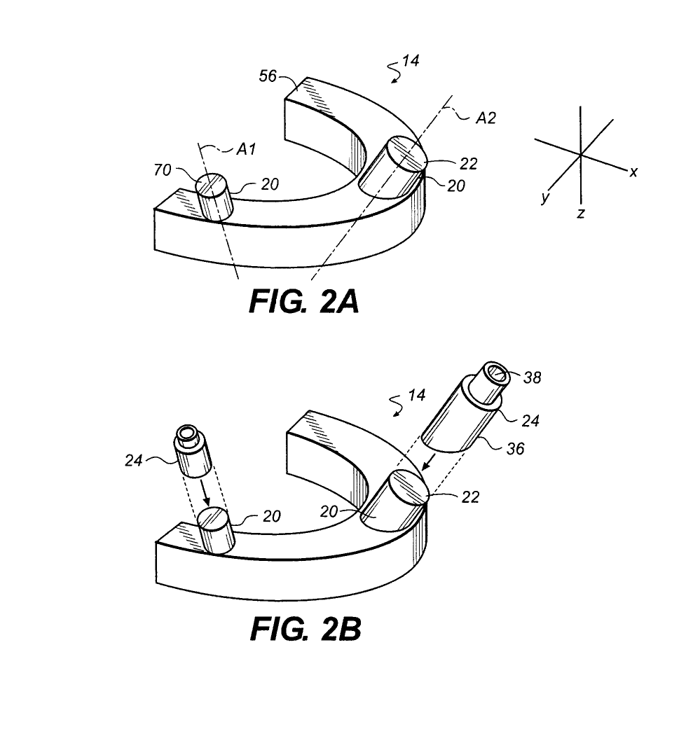

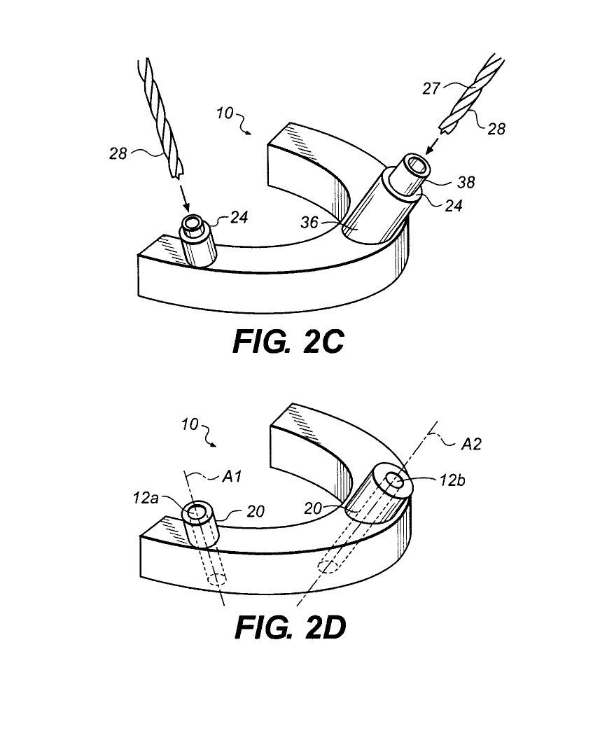

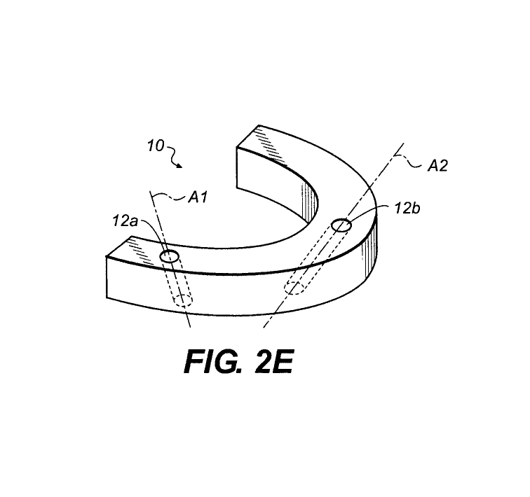

Embodiments of the present invention address the problem of fabricating a surgical guide on a 4-axis milling apparatus by forming guide features that extend outward from one or more surfaces of the surgical guide and then seating drill guide sleeves to provide hole guides for manual drilling of guide holes. Referring to FIGS. 2A-2E, there is shown a sequence for surgical guide 10 fabrication using this approach. Implant planning for surgical guide 10 to suit an individual patient can be performed using conventional methods or can use a 3-D volume image of the patient's mouth structure, such as using a computed tomography (CT) or cone-beam CT (CBCT) image, for example. The implant plan can be generated with the assistance of appropriate software using the obtained volume image.

For guide fabrication in FIG. 2A, a milling apparatus, such as one of the units described subsequently, is used to form an intermediate structure 14 shaped for conformance to the dental arch of a patient and having two protruding guide features 20 machined, or otherwise formed to shape, on one or more surfaces 56. Each guide feature 20 is aligned about a corresponding drill axis, shown in FIG. 2A as exemplary drill axes A1 and A2 and has a planar surface 70 that is normal to the drill axis, that is, normal to the length of the drilled hole. It can be observed that guide features 20 can be formed using a 4-axis milling apparatus; a 5-axis machine could also be used, but the additional capability, complexity, and cost are not required for this fabrication. Guide features 20 are cylindrical in the embodiment shown and tilted at an oblique angle relative to the milling apparatus axes shown in FIG. 2A, but may have any suitable shape for performing the needed functions to guide drilling operation.

FIGS. 2B and 2C show the seating and use of a drill guide sleeve 24 onto each guide feature 20. Drill guide sleeve 24 has a seat portion 36 that fits over guide feature 20 and a shaft guide portion 38 that guides a shaft 27 of a fabrication drill bit 28 for drilling into surgical guide 10 at the intended angle. FIG. 2D shows surgical guide 10 at the completion of this fabrication sequence, with guide holes 12a and 12b extending through this surgical appliance. Guide ho les 12a and 12b are not drilled by the 4-axis milling apparatus but are manually drilled. FIG. 2E shows surgical guide 10 following an optional step in which one or more guide features 20 are reduced in height, re-dimensioned, re-shaped, or even removed in preparation for placement into the patient's mouth.

Protruding guide features 20 as shown in FIGS. 2A, 2B, and 2D can have multiple purposes. First, during the milling of the surgical guide 10, guide features 20 can guide the manual drilling of tilted guide holes 12a and 12b (out of the yz plane) during the manufacture of the surgical guide 10. Once surgical guide 10 is fully manufactured, the dentist can further employ guide feature 20 during surgery. During surgical procedure, the dentist drills one or more holes into the patient's jawbone to insert an implant of defined length. Proper dimensioning and angle of guide features 20 help in surgical drilling and guide the dentist in drilling holes at the correct angle and depth.

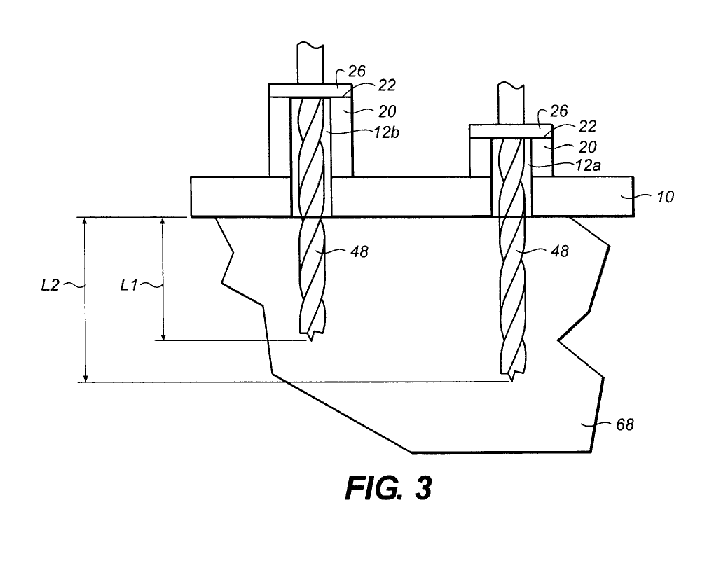

As shown in FIG. 3, collar 26 used on surgical drill bit 48, abuts on the planar surface of the protruding guide feature 20 and, by fixing the usable length of surgical drill bit 48, sets the depth of the hole that will be drilled into the patient's jawbone 68. The guide hole 12a or 12b has been drilled at the proper angle using the technique previously shown in FIGS. 2A-2E with manual drilling, or with similar fabrication. As shown in FIG. 3, the height of guide feature 20 can also be used as a depth guiding feature for setting a variable length, such as those shown as lengths L1 or L2. The variable length depends on the required depth of the implant mounting features, typically defined by dedicated software tools that help to generate the implant plan, as noted previously in the background section. The planar surface of guide feature 20 is normal (perpendicular) to the length direction for the drilled hole 12a or 12b. A planar surface 22 of guide feature 20 can be oblique with respect to the milling apparatus axes (FIG. 2A).

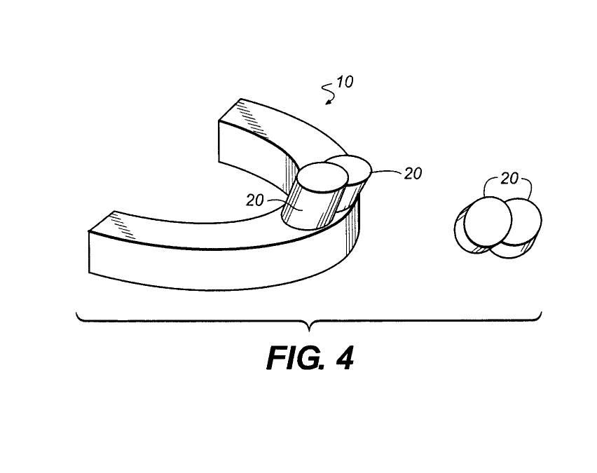

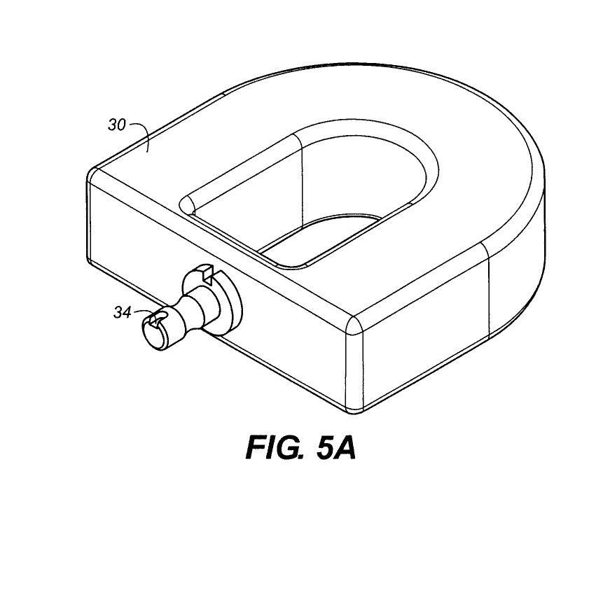

FIG. 4 shows an alternate embodiment having two guide features 20 that are very close together or touching. For such cases, drill guide sleeve 24 (FIG. 2B) may need to be modified to allow drilling of a hole in each guide feature 20.

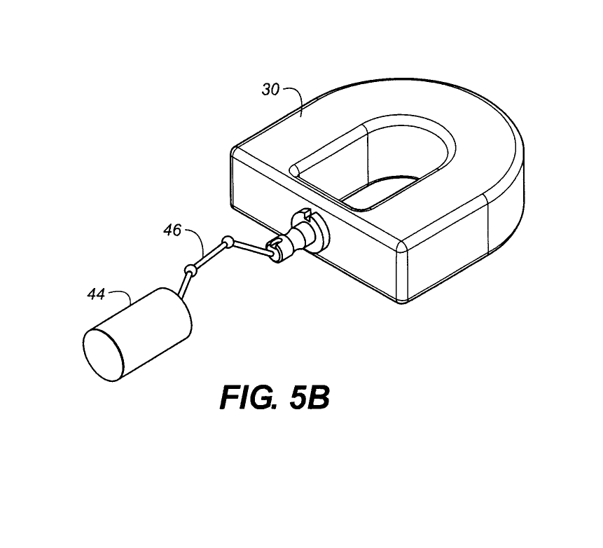

A number of alternative embodiments of the present invention assist to enable and simplify fabrication of surgical guide 10 using a 4-axis milling apparatus. FIG. 5A shows a workpiece 30 attached to a spindle 34 prior to machining operation. There can be a number of different arrangements used for spindle coupling.

The perspective view of FIG. 5B shows an alternate embodiment using a carrier 46 for mounting workpiece 30 in a 4-axis machine. By providing a swivel axis arrangement on a chuck 44, the surface presented to the tool using carrier 46 can be tilted at an oblique angle with respect to multiple axes. Here, carrier 46 rotates the workpiece to positions spaced apart from the axis of rotation. This type of arrangement thus effectively provides a measure of 5-axis capability for surgical guide fabrication.

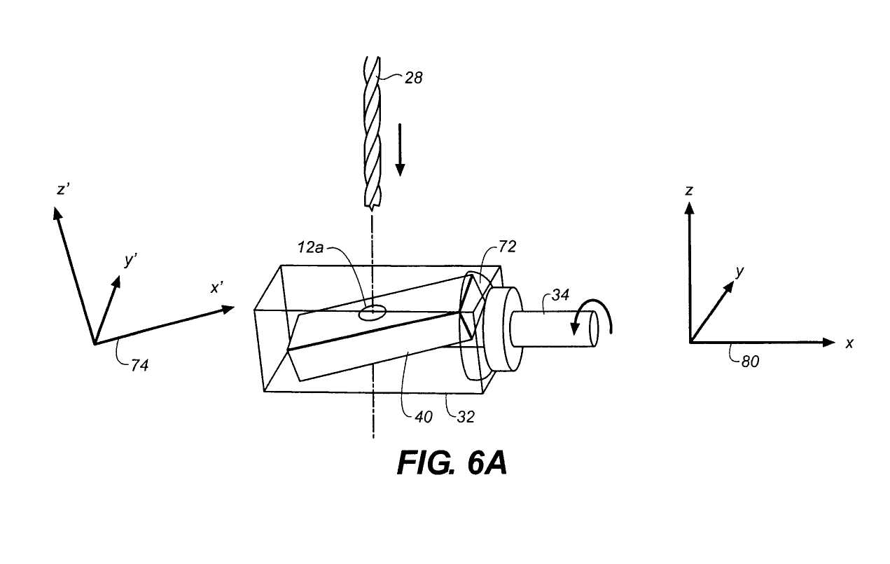

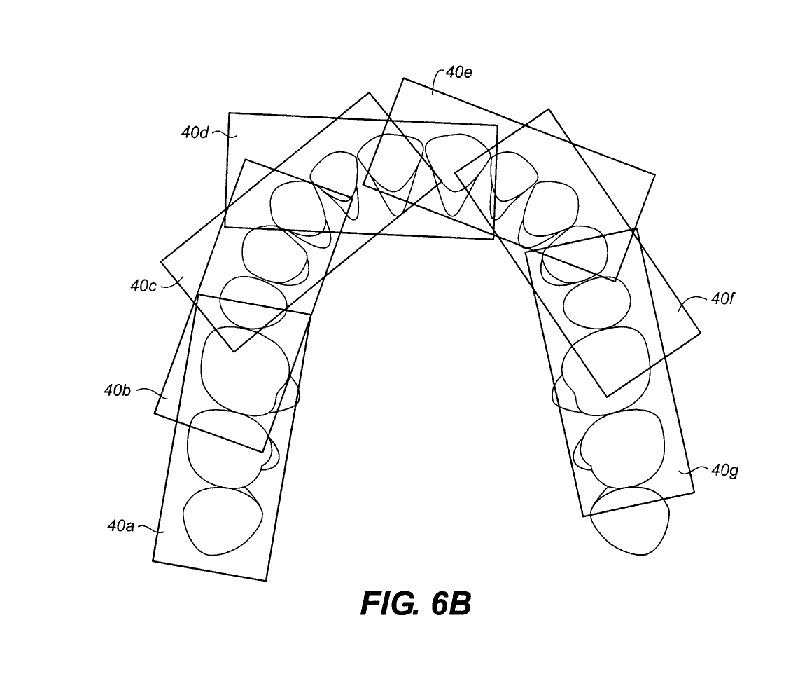

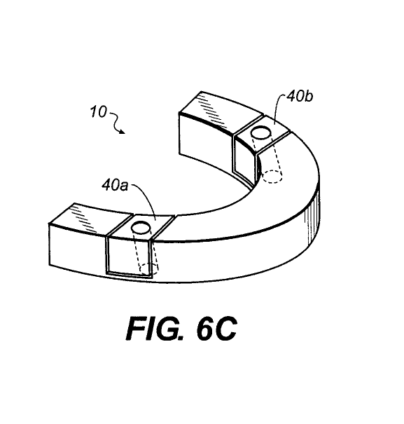

The perspective view of FIG. 6A shows an alternate embodiment for supporting and milling a workpiece 32 for forming a partial surgical guide 40 that is designed to span only a few teeth rather than the full dental arch. As previously described, the 4-axis milling apparatus comprises a spindle 34 that can rotate about an x axis, a machine axis 80, and a drill bit 28 that can drill holes along a z direction. Starting from a workpiece 32, the milling apparatus is able to mill a structure 40 having at least a planar surface while keeping a clearance 72 to maintain the contact between the structure and the spindle 34. At least one surface of the structure 40 is tilted relative to the x axis of the apparatus. Considering an axis reference (x′, y′, z′) 74 with (x′, y′) defining the plan of the tilted surface of the structure 40, the x axis of the 4-axis apparatus is not parallel to the (x′, y′) plane.

Drill bit 28 can then drill hole 12a through structure 40 along the z-axis of the machine. This hole is not parallel to the (y′z′) plane in the axis reference (x′, y′, z′) 74 of structure 40. This method then allows the drilling of some holes directed out of an (y′z′) plane of structure 40 using a 4-axis milling apparatus, though the 4-axis milling apparatus is not able to drill holes out of its own (y,z) plane. A hole tilted out of the (y′z′) plane of the structure can then be drilled without any manual drill step.

FIG. 6B shows a plan view with multiple partial surgical guides 40a, 40b, 40c, 40d, 40e, 40f, and 40g that can be individually positioned over local areas of the dental arch. In practice, as noted previously, holes not in the yz plane are drilled separately from the milling operation.

In an alternate embodiment of the present invention, shown in FIG. 6C, a horse-shoe shaped surgical guide 10, conformal to the patient's dental arch, can be provided as a housing for holding one or more partial surgical guides 40a, 40b having tilted holes that are oblique to surfaces of surgical guide 10. In this way, a single guide 10 can be used to more independently position two or more partial surgical guides 40a and 40b, each positionable within the housing and having a hole of the required angle for drilling into the patient.

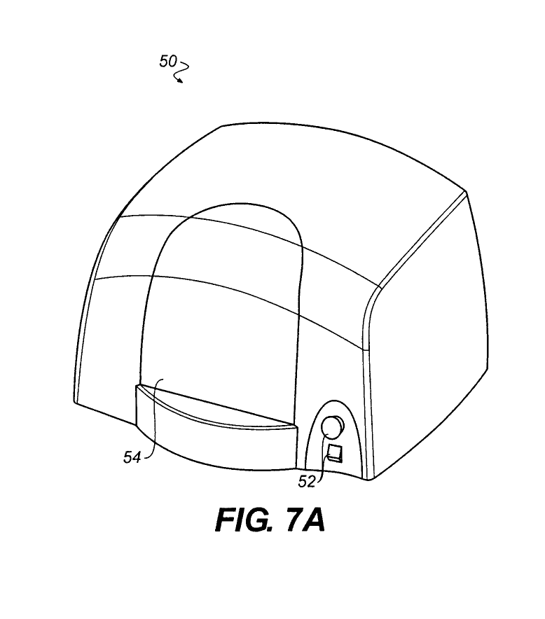

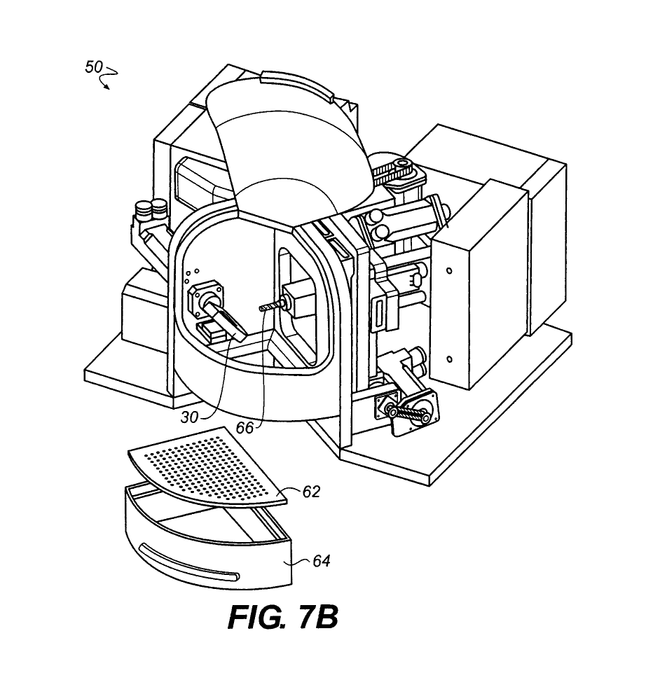

FIG. 7A shows a milling apparatus 50 that can be used for surgical guide fabrication in a dental office or other facility. Apparatus 50 is preferably small-scale to provide a small footprint at the dental office or other facility. Milling apparatus 50 obtains data about the patient for implant planning that defines intermediate structure 14 or partial surgical guide 40 and performs the fabrication steps to automatically generate intermediate structure 14 (FIG. 2A, 2B) or surgical guide 40. According to an embodiment of the present invention, this data is obtained from a volume image of the patient and used to generate an implant plan. An implant plan shows the position of a planned implant relative to patient features. Additional information about the surgery type is also obtained and used for generating the implant plan. Apparatus 50 has a protective opening/door 54 and controls 52 for initiating operation and reporting process completion or error. The partially exploded view of FIG. 7B shows internal components of milling apparatus 50, including a filter 62 and a water tank 64. The relative positions of workpiece 30 and a tool 66 are also shown by way of example.

According to an embodiment of the present invention, surgical guide 10 is formed from a plastic material such as PMMA (Poly(methyl methacrylate)) or other plastic. Other machinable materials can also be employed for forming surgical guide 10.

According to an alternate embodiment, holes drilled in surgical guide 10 are temporarily filled with a solid block of radio-opaque material, such as gutta percha, as markers and a volumetric x-ray scan is obtained with the surgical guide in the mouth of the patient. The radio-opaque markers indicate the position of prosthetic teeth that will be fixed on an implant following osteotomy and implantology surgery. Prior to surgery, the dentist can check that holes in the surgical guide 10 are in the correct position and have the correct tilt, using visualization of the opaque elements. The radio-opaque material is removed following the volumetric scan.

1. A method for forming a surgery guide for osteotomy, comprising:
shaping a block of material for conformance to at least a portion of the dental arch of a patient;
shaping at least one guide feature that protrudes from a surface of the shaped block material;
seating a drill guide sleeve on the at least one guide feature; and
guiding the shaft of a drill into the sleeve and drilling a hole through the shaped block material.

2. The method of claim 1 further comprising removing the drill guide sleeve and re-shaping the at least one guide feature after drilling the hole through the shaped block material.

3. The method of claim 1 wherein shaping the at least one guide feature comprises forming a cylindrical structure using a milling machine.

4. The method of claim 1 wherein shaping the at least one guide feature comprises forming a cylindrical structure that is tilted at an oblique angle relative to any of the three orthogonal axes of a milling apparatus.

5. The method of claim 1 wherein the shaped guide feature comprises a planar surface that is normal to the length of the drilled hole.

6. The method of claim 3 wherein the milling machine is a 4-axis milling machine.

7. The method of claim 5 wherein the planar surface is oblique with respect to any axis of the milling machine.

8. The method of claim 1 wherein the drill guide sleeve fits around the at least one guide feature.

9. The method of claim 1 further comprising obtaining information about the patient from a volume image and generating an implant plan according to the obtained information.

10. A method for forming a surgery guide for osteotomy, the method comprising:
shaping a block of material for conformance to at least a portion of the dental arch of the patient, using a 4-axis milling apparatus;
shaping at least one guide feature that protrudes from a surface of the shaped block material, wherein the at least one guide feature is a cylindrical structure that is tilted at an oblique angle relative to a plane that is orthogonal to an axis of rotation of the milling apparatus;
seating a drill guide sleeve on the at least one guide feature, wherein the drill guide sleeve includes a seat portion that fits over the at least one guide feature and a shaft guide portion that guides a drill bit in a direction that leads into the at least one guide feature; and
drilling a hole through the shaped block material by a drill shaft guided into the shaft guide portion.

11. The method of claim 10 further comprising:
filling the hole in the shaped block material with a radio-opaque material;
performing a volumetric scan with the filled shaped block material in the mouth of the patient; and
verifying alignment of the the hole using the volumetric scan.

12. The method of claim 10 further comprising generating an implant plan from a volume image of the patient.

13. A surgical guide for osteotomy, the guide comprising at least one protruding guide feature having a seat for mating with a surgical drill guide collar disposed on a drill bit.

14. (canceled)

15. A method for mounting a removable workpiece to a spindle in a 4-axis milling machine, the milling machine having an axis of rotation, the method comprising:
coupling the workpiece to a carrier having swivel portions;
attaching the carrier to the spindle; and
using the carrier to rotate the workpiece relative to the axis of rotation.

16. A method for forming a surgery guide for osteotomy, comprising:
shaping a block of material for conformance to at least a portion of the dental arch of a patient;
shaping at least one guide feature that protrudes from a surface of the shaped block material;
seating a removable drill guide sleeve on the at least one guide feature, the drill guide sleeve including a shaft guide portion having a drill axis;
guiding a shaft of a drill bit along the drill axis of the shaft guide portion when the drill guide sleeve is seated on the at least one guide feature; and
forming a guide hole through the shaped block material.

17. A method for forming a surgery guide for osteotomy, comprising:
using a 4-axis milling machine to shape a block of material for conformance to at least a portion of the dental arch of a patient;
forming at least one guide feature that protrudes from a surface of the shaped block material;
seating a removable drill guide sleeve on the at least one guide feature, the drill guide sleeve having a drill axis; and
guiding a shaft of a drill bit along the drill axis into the drill guide sleeve to manually drill a guide hole through the shaped block material.

18. The method of claim 10 further comprising:
removing the drill guide sleeve; and
re-shaping the at least one guide feature.

19. The surgical guide of claim 13 wherein the at least one protruding guide feature has either (1) a cylindrical cross-sectional shape or (2) a planar surface normal to a drill axis of the drill bit

20. The surgical guide of claim 13 further comprising:
a base for conformance to at least a portion of a dental arch of a patient; and
the at least one protruding guide feature protruding from a surface of the base onto which the surgical drill guide collar can be seated.
